(19) United States
(12) Patent Application Publication
(10) Pub. No.: US 2006/0188580 A1
(43) Pub. Date: Aug. 24, 2006

(54) Anti-microbial granules

(76) Inventor: Ralph Sacks, Rosemont, IL (US)

Correspondence Address: WELSH & KATZ, LTD, 120 S RIVERSIDE PLAZA, 22ND FLOOR, CHICAGO, IL, 60606, US

(21) Appl. No.: 11/347,171

(22) Filed: Feb. 3, 2006

Related U.S. Application Data
(60) Provisional application No. 60/649,746, filed on Feb. 3, 2005.

(51) Int. Cl. A61K 33/24; A61K 33/34; A61K 33/38; A61K 33/32

(52) U.S. Cl. 424/489; 424/618; 424/617; 424/649; 424/638; 424/641

(57) ABSTRACT

Antimicrobial granules comprising granular materials coated with antimicrobial metal agents for use in various surface coating and grouting materials are provided. The antimicrobial granules have utility in imparting durable, safe, inexpensive and powerful antimicrobial properties to materials into which they are incorporated, such as epoxy coating and grouts for surgical theaters, public washrooms, and food processing plants. The granules further are capable of imparting a timed-release dosage of antimicrobial agents so that the effectiveness of the coating is palpable for extended periods.

### RELATED APPLICATIONS

This application claims priority from Provisional Application No. 60/649,746 filed Feb. 3, 2005, which is hereby incorporated in its entirety.

### FIELD OF THE INVENTION

The present invention relates to granulated additives to grout, mortars, concrete and other cement and coating mixtures. More particularly the present invention relates to anti-microbial granules for use in the making of cements and coatings that when used provide long term antimicrobial effects for floors, walls and other surfaces.

### BACKGROUND OF THE INVENTION

Many surfaces in homes, health care facilities, food service and food preparation facilities cleaned or treated with a disinfectant nevertheless remain contaminated with living microorganisms. Such microorganisms may be pathogenic, may damage the surface, cause odor, and/or provide a reservoir of contamination that damages products or processes that come near the surface. As a result, frequent cleanings or treatments with powerful anti-microbial agents, including bleach, heat, and radiation, are necessary to reduce or eliminate the micro-organisms. This is the typical way hygienic conditions are maintained in for example, a meat packing facilies, surgical operating theater and/or household bathrooms and public facilities. In some cases surface or material damage can be structural and/or aesthetic, such as mildew growing into bath caulking or under paint.

It is well known in the art that present day methods of cleaning and disinfecting surfaces and objects are not completely effective and that some microbes survive the most thorough of cleanings. In the case of bacteria, the doubling time (time it takes for one cell to divide into two cells) can be as short as 20 minutes. The exponential nature of growth means that surfaces will again have significant numbers of microorganisms after just a few hours time. Further, organisms trapped under a coating can thrive because disinfectant agents, even ultraviolet radiation and bleach, do not penetrate coatings and objects very well. In this manner microorganisms that survive a treatment can flourish between cleaning treatments.

One approach to this problem has been to incorporate anti-microbial agents into paints, caulking or other coatings applied to a surface with the goal of retarding microbial growth between cleanings. It has been found, however, that chemicals used for this purpose, such as triclosan or anti-mildew fungicides, have several disadvantages.

The places where a surface having antimicrobial properties would be most advantageous are the places where hygiene is also most important and are often times the most frequently washed places. However, antimicrobial organic chemicals are known to be smaller molecules and consequently are typically leached out of the coating or surface at a rate that is increased where the surface is washed frequently. As a result the antimicrobial organic chemicals of prior art treatments are lost most rapidly in the very environments in which they are most important such as in public washrooms and meat packing facilities.

In addition, many antimicrobial organic chemicals are known to introduce adverse health risks in and of themselves. Common additives, used in previous antimicrobial treatments, have included chlorothalonil (a U.S. E.P.A. category B2 probable human carcinogen; no longer available), 3-iodo-2-propynyl carbamate (U.S. E.P.A. category I, Highly Toxic), thiabendazole (carcinogenic and developmental toxin), and the like. Clearly, it would be beneficial if the anti-microbial treatments used in such processes were not themselves human health hazards.

Antimicrobial organic chemicals, triclosan in particular, are suspected of giving rise to chemical resistance in bacterial populations, just as chronic antibiotic drug usage results in antibiotic-resistant bacteria. For this reason, some floor coating manufacturers have stopped using this additive in food processing plant resin floor coatings.

Lastly, antimicrobial organic chemicals used as antimicrobial additive may not withstand harsh conditions used for routine cleaning or during a particular process. Depending of the nature of the surface, cleaning processes can include the use of 98% pure sulfuric acid, autoclaving, ultraviolet radiation, or ethylene oxide, each of which is known to inhibit or destroy the antimicrobial properties of prior art antimicrobial organic chemicals.

One attractive class of antimicrobial agents that can overcome these weaknesses are inorganic antimicrobials, in particular metals such as silver, copper, zinc, tin, gold and titanium. These, and other metals known in the art, exert powerful antimicrobial activities in concentrations that are not harmful to humans. Silver and copper in particular have been used as antimicrobial agents since Roman times. However, large amounts of these metals can be prohibitively expensive, and current metal-containing antimicrobial agents have drawbacks that reduce their efficacy particularly when incorporated into coatings.

In direct contrast to organic chemical antimicrobial additives known in the art, the metal-based antimicrobial agents do not readily leach out of the surface and are highly resistant to heat, radiation, and chemical attack. However, tests have shown that neither antimicrobial metals nor zeolite antimicrobial metal agents, such as are commercially available from AgION™ Technologies, Inc. Wakefield, Mass., exert adequate antimicrobial effect when mixed directly into coatings. It is believed that the metal or metal-containing antimicrobial agents in the prior art either float on top of or sink to the bottom of for example, uncured fluid coatings, and/or become totally encapsulated by the surrounding medium and unavailable to exert an antimicrobial effect. It is also believed that the antimicrobial metal agent on or close to the surface of the object or coating is rapidly eroded. It is seen, then, that antimicrobial metal agents known in the art are not ideally adapted to application onto surfaces or incorporation into objects or coatings required in e.g., hygienic facilities, such as epoxy coatings and grouts.

There exists a need for coatings with durable, inexpensive, effective, and safe antimicrobial properties. There is also a need for antimicrobial materials that can be added to mixtures that are used as coatings or formed into objects, such as concrete, epoxy, plastic, and the like for applications such as floors, shelves, countertops, other work surfaces or other places where antimicrobial activity is desirable.

There is also a significant need for antimicrobial materials that can be used to conveniently and economically impart microbial resistance to surfaces of existing objects as easily as a new coat of paint can impart improved aesthetics.

### DETAILED DESCRIPTION OF THE INVENTION

While the present invention is susceptible of embodiment in various forms, there is discussed herein a number of presently preferred embodiments that are discussed in greater detail hereafter. It should be understood that the present disclosure is to be considered as an exemplification of the present invention, and is not intended to limit the invention to the specific embodiments discussed. It should be further understood that the title of this section of this application (“Description of the Invention”) relates to a requirement of the United States Patent Office, and should not be found to limit the subject matter disclosed herein.

This application discloses granular materials coated with antimicrobial metal agents for use in continual effective disinfection. Because the metals themselves are not released at rates or levels toxic to humans, antimicrobial granules of the invention are safe for human work and living surfaces while imparting durable, long lasting anti-microbial properties to surfaces and objects into which they are incorporated.

Granules amenable to coating according to the invention include but are not limited to: silica granules (such as silica sand, glass particles, ground quartz and the like), perlite, aluminum oxide, carborundum, talc, metal silicates, rubber granules, aluminum and other metal granules, ceramic granules, carbon granules, polymer granules, stones and any other granular or powder material that can be used as an additive or filler in, stucco, paints, epoxy-based materials such as coatings, sealants, flooring, tile grouting or wall system or poured, molded or cast concrete, plastic or epoxy surfaces, objects or materials and the like. Persons having ordinary skill in the art will understand that the term granules, therefore is given its broadest definition here and should not be limited to only those materials and forms enumerated here. Granules, particulates, fibers, sands, chips and other such terms can be used interchangeably without departing from the novel scope of the present invention.

The granules are often chosen to impart attributes of the granules themselves to the surface, coating or object with which they are incorporated. For example, sand and similar granules are used to impart anti-slip texture to a floor coating such as a paint or an epoxy floor coating. Carbon fibers can impart both tensile strength and electric conductivity. As is known in the art, carbon, copper, silver, nickel, aluminum and other metal granules can be added to epoxy or other coatings or objects to dissipate static electricity or reduce electromagnetic interference with electronics.

It will be understood by those having ordinary skill in the art that the antimicrobial granules of the invention generally retain their utility as fillers or texturizers in stuccos, paints, epoxy-based materials such as coatings, sealants, flooring, tile grouting or wall system or poured, molded or cast concrete, plastic or epoxy surfaces and objects and the like that are characteristic of the granule material. It will also be understood that the antimicrobial metal agent, a binding agent or the coating technique used can modify the overall properties of the antimicrobial granules relative to the starting granules without departing from the novel scope of the present invention. For example, silica sand coated with electricity-conductive materials can impart electrical conductivity to materials in which they are added as fillers.

It will be understood by persons having ordinary skill in the art that no limitation in particle size and shape is intended in the antimicrobial granules of the present invention, as different applications will require different types and/or sizes of granules. For example, if a rough surface is required, large (up to about 6 mm diameter) granules can be used according to the invention; where mechanical strength is required, granules can be very elongated (for example: glass slivers or flakes, carbon fibers, Kevlar, and/or other natural or synthetic polymeric fibers).

Essentially, any minimum granule size of the invention will be dictated by the nature of the coating process and the anti-microbial agent used. Where, for example, the antimicrobial metal agent is itself particulate such as a metal-containing zeolite (as are commercially available from AgION Technologies, Inc. Wakefield, Mass.), or metal-containing glass particles (commercially available as Ionpure® from Ishizuka Glass Co., Japan), the diameter of the granules to be coated should be at least twice the diameter of the particles of antimicrobial agent.

It will also be understood by persons having ordinary skill in the art that the granule sizes and types noted herein are examples and other effective granules and particulates can be utilized effectively without departing from the novel scope of the present invention.

The invention disclosed herein further includes the method of coating granular material with a metal-based antimicrobial agent, anti-microbial granular materials that have metal-based anti-microbial coatings, and use of anti-microbial granular materials to impart anti-microbial properties to materials containing the antimicrobial granules.

As used herein, antimicrobial metal agent and the synonymous term metal-containing antimicrobial agent can mean a metal having antimicrobial activity or an agent comprising a metal having antimicrobial activity. The antimicrobial metal agent can be particulate. The metal-based antimicrobial agent applied to the granules can be comprised of silver, copper, tin, zinc, gold, titanium, and other metals as are known in the art to have antimicrobial activity. The metal-based antimicrobial agent can be the elemental (uncharged) metal or an oxidized and cationic form thereof, such as metal oxides, salts and complexes capable of releasing a metal cation to exert its antimicrobial activity. Herein, an oxidized metal means only metal in cationic forms as in metal salts, oxides and the like.

As used herein, the terms coat, coating, and coated refer to the physical association of an antimicrobial metal agent with a granular carrier. As will be understood by those having ordinary skill in the art, the required coating association can be accomplished by any of numerous mechanical, chemical, electrical or electrochemical coating methods that achieve the required deposition and physical association of the antimicrobial agent onto the granules, without departing from the novel scope of the present invention.

As described herein, granule coating by adhesive or binding agents is preferred where a particulate antimicrobial metal agent is used. It will be understood that in some cases the anti-microbial agent may not exist solely on the exterior of a coated granule, but can partially or totally permeate the granule, for example in perlites.

In some embodiments, granules are coated with an uncharged antimicrobial metal by mechanical or physical actions such as by tumbling or vapor deposition, electrical methods such as sputtering, and numerous other methods of coating objects with metal as are known in the art. In other preferred embodiments, the antimicrobial metal agent is deposited onto the granules as a metal salt or metal ion-containing complex by evaporation, precipitation, polymerization, mechanical accretion, action of adhesives and/or other manners, all of which are well understood in the art.

In a preferred embodiment, the antimicrobial agent is comprised of antimicrobial metal associated with a substrate capable of slowly releasing the antimicrobial metal over a long period of time. In a further preferred embodiment, the antimicrobial agent comprises an antimicrobial metal cation associated with an ion-exchange medium.

For the purposes of this invention, an ion-exchange medium is a material from which a antimicrobial metal cation can be released into the environment when other cations replace the metal ion from its association with the ion-exchange medium or by dissolution of the medium with concomitant release of the antimicrobial metal cation. By this definition, antimicrobial metal cation-containing glass and hydroxyapatite, as disclosed in U.S. Pat. Nos. 6,482,444 to Bellantone et al., 5,290,544 to Shimono, et al., and 5,009,898 to Sakuma et al., (each incorporated herein in their entirety by reference) are all ion exchange materials, even though these materials may not necessarily fit within the usual understanding of the term ion-exchange material.

In further preferred embodiments, the antimicrobial agent comprises a metal associated with a zeolite capable of acting as an ion exchange medium. In an especially preferred embodiment, the metal is silver associated with a zeolite. In a further preferred embodiment, the silver-containing zeolite is the AgIon™ antimicrobial available from AgIon™ Technologies, Wakefield, Mass.

In another preferred embodiment, the metal ion is associated with a silicate glass that acts as an ion-exchange medium. In a further preferred embodiment, the antimicrobial metal agent is Ionpure® from Ishizuka Glass Co., Japan, available in the US from Marubeni America, Santa Clara Calif. This antimicrobial metal agent, comprised of soluble glass made with silver oxide, is particularly useful where clear optical qualities are important, such as where the color or transparency of the granules is to be maintained. Relative to the AgIon™ antimicrobial product, the Ionpure® product can also release antimicrobial metal ions more slowly in very moist, high ionic strength environments.

Another preferred antimicrobial metal agent comprises a traditional cation-exchange material, such as a cation-exchange resin, and an antimicrobial metal cation.

Technology for coating granules is well known in the art. One preferred method of attaching the antimicrobial agent to granules is to put the granules to be coated into a revolving drum. In many instances a mortar mixer or concrete mixer is used.

Preferred adhesives for coating granules with antimicrobial metal agents include epoxies, polyesters, synthetic and natural latexes, acrylics, olefins, vinyls, methacrylates, and urethanes. In a preferred embodiment, epoxy is the adhesive. See the Examples, below. In many cases, epoxy-based adhesives are well-suited and preferred, but it will be evident to those skilled in the art that other materials can be used as adhesives or binders, even if the material is not generally thought of as an adhesive, such as glass or mineral powders that can be used in a baked enamel-type coating process. NEOCAR™ Acrylic 850 and other NEOCAR® latexes and especially NEOCAR® acrylic latexes (Dow Chemical Company, Midland Mich.), UCAR™ Latex DT 250 and other UCAR™ vinyl and styrene butadiene latexes and especially UCAR™ styrene acrylic latexes (Dow Chemical Company, Midland Mich.), the EPON series of epoxy resins (Resolution Performance Products, Houston, Tex.), Derakane® 411 resin and other Derakane® epoxy vinyl ester coatings (Ashland Composite Polymers, Dublin Ohio), and many other adhesives and coatings are useful for coating granules with antimicrobial metal agents according to the teaching of the present invention without going beyond the scope thereof.

Also a coating process may not require any adhesive or binder as such. Coating can be accomplished by deposition of metals, metal-containing zeolites, metal salts, metal-containing hydroxyapatites or other metal-containing compounds or component by electric charge, electrochemical methods, evaporative methods, baking, fusing, vapor-deposition, sputtering or techniques known in the art.

As will be understood by persons having ordinary skill in the art, the absolute amount of metal antimicrobial agent attached to individual granules can vary within any preparation, and the distribution of antimicrobial agent among granules will depend on the coating technique used. The antimicrobial granules of the invention preferably, but might not, have uniform or similar amounts of antimicrobial agent attached to individual granules. As would be expected by those having ordinary skill in the art, some individual granules created using the teachings of the present invention can have no antimicrobial agent attached whatsoever. Nor does the presence of conglomerates of coated granules remove the granules from the novel scope of the invention.

Where the granule coating includes a mechanical mixing step, the mixer size and speed will depend on the quantity and type of granules to be coated, as will be evident to those of skill in the art. In some cases, pre-tumbling or premixing can be desirable to for example remove sharp corners from silica sand granules and to disperse the antimicrobial metal agent among the granules prior to addition of an adhesive or binder. Tumbling parameters and other pre-treatment techniques are known in the art.

In a preferred method, granules are added to a mixer and the mixer is activated. Subsequently, an effective amount of a particulate anti-microbial metal agent such as an amount of about 0.1% by weight to about 30% by weight of the granules is added while the mixer is turning. After the mix has tumbled for a few minutes, an adhesive is added. Epoxy liquids, comprised of a component A that includes a resin adapted for use in the conditions in which the antimicrobial granules are to be used, and a component B that includes the hardener, are added into the mixer. Mixing continues until the epoxy hardens. It will be understood by persons having ordinary skill in the art that the amount of time of mixing at each stage is dependent on many factors, including materials, quantities, effectiveness of the mixer, and others, all of which are within the novel scope of the present invention.

As is plain to those having ordinary skill in the art, the ratio of granules to antimicrobial metal agent will also inversely depend on the size of the granules. Consequently granules with especially high surface are per mass (such as very fine, fibrous or flake shaped granules) or low weight per volume (such as perlite) can require even about a three to one by weight ratio of agent to granule.

Epoxies and other adhesives can be chosen to withstand, for example, acidic materials such as 98% sulfuric acid, or solvents, such as methylene chloride. A person having ordinary skill in the art will be able to properly formulate an adhesive adapted to the environment in which the granules will be used. Alternatively, the epoxy or other adhesive is added to the granules before the anti-microbial agent is added to the mixer. In an especially preferred embodiment, mixing continues until such time as that the epoxy causes the anti-microbial powder to adhere to the granules while the granules do not stick to each other and form large conglomerates. The tumbling or mixing continues until such time as the epoxy is set, a common time being from about 20 minutes to about 6 hours. The time of overall tumbling depends on the kind of epoxy or other adhesive used, as is understood by those having ordinary skill in the art.

Setting of certain epoxies and other adhesives requires or can be hastened by heat, and so the mixer can be heated in ways known to those having ordinary skill in the art. Other additives, such as coloring agents and tints, can be applied to the granules so that the resulting granules impart both color and antimicrobial properties to the material in which they are incorporated. It will be understood by persons having ordinary skill in the art, that for certain applications, the granules can be pre-coated with a dye or colorant and subsequently coated with the antimicrobial agent in separate coating steps. In other applications the other additive can be mixed with the antimicrobial agent and applied in the same coating process.

Metal-based antimicrobial agents do not readily leach out of the surface and are highly resistant to heat, radiation, and chemical attack, in direct contrast to organic chemical antimicrobial additives known in the art. However, tests have shown that neither antimicrobial metals nor unattached metal-containing zeolite antimicrobial agents exert adequate antimicrobial effect when mixed directly into coatings. It is believed that metal or metal containing antimicrobial agents not attached to granules either float on top of or sink to the bottom of for example, uncured fluid coatings, and/or becomes totally encapsulated by the surrounding medium and unavailable to exert an antimicrobial effect.

The granules of the present invention, however, will often be large enough that there is adequate exposure of the metal to the environment so the slowly-released metal can exert its antimicrobial effect.

It is useful, but not an essential element of the invention, that the granules are designed to be evenly distributed within the medium to which they are added, so they are sequentially exposed as the surface is worn away or granules lost from the surface. It is also useful, but not essential, that new granules are exposed to the environment as the surface is worn away so new antimicrobial metal agent is available as the granules are themselves expoed.

The antimicrobial granules according to the present invention are admixed into the composition in an amount effective to provide a desired amount of antimicrobial protection to the composition. Typical amounts are about 1% to about 500% by weight of the binder, adhesive, resin or polymer used in the coating or object. Where the resin or polymer is particularly expensive, the higher amount is useful to reduce the overall cost of the applied coating or the object.

For any particular application, however, it will be apparent to one of skill in the art that the inventive antimicrobial granules can be mixed with many alternative cements, polymeric resins and/or coatings and the like for which antimicrobial properties are desirable. Among these are acrylics, latexes, olefins, silicones, Portland cement, methacrylates, vinyl esters, and other spreadable or flowable coating or forming materials as are known in the art for application to surfaces or fabrication into objects, each of which can be used equally or more appropriately in a particular circumstance than others, while benefiting from incorporation of the antimicrobial granules of the invention.

It will be recognized that the granule coating system will frequently comprise the same bonding materials as the mix into which they are incorporated in the final use. That is, formulation simplicity and compatibility will frequently lead one of skill to use latex adhesives for granules destined for addition to latex-based coatings because compatibility is assured. However, one of skill in the art will also know that disparate granule coating technologies do not preclude addition of the granules.

Epoxy resin-based materials are particularly resistant to mechanical and chemical attack, making them advantageous for harsh environments or where the surface is to remain in serviceable condition indefinitely. The antimicrobial granules of the present invention are well-suited for incorporation into epoxy-based materials. They provide not only enduring antimicrobial attributes, but can be used in large amounts as filler, such that as little as one gallon of epoxy can be mixed with four gallons of granules. When so used, the cost of the final surface, object, or material is markedly reduced relative to unfilled epoxy, because epoxy is generally much more expensive than the granules of the present invention.

The consistencies of epoxy materials containing the inventive antimicrobial granules can range, prior to setting, from freely fluid and hence applicable with a sprayer or roller, to textured like stucco, to thick, like putty or concrete and hence applicable with a trowel. These materials can be used to coat any surface, interior or exterior, including walls, ceilings, counters, shelves, etc., and in some applications will comprise the entire object, such as epoxy resin-based laboratory work surfaces. Other polymers, coatings and cements incorporating the granules according to the invention will similarly have varying consistencies adapted to the specific mode of forming or application.

In a preferred embodiment, the antimicrobial granules according to teachings of the present invention are mixed with a two component epoxy system comprised of Part A and Part B. For two component epoxies, common usage of these terms is that Part A comprises the epoxy resin and Part B comprises the hardening agent. One such system is described in U.S. Provisional Application No. 60/648,179. Depending on the consistency and desired thickness of coating, different epoxy resins or additional solvents and filler materials can be added. For example, to form a thick layer of epoxy flooring, the mix is typically spread with a trowel onto an existing floor to roughly a ¼ inch thickness. Depending on cost and other factors, the thickness is varied and can be as thick as several inches to form the full slab floor (with attendant structural elements as are know to persons having skill in the art). Where anti-slip texturing is desired, rough anti-microbial granules can be applied to the surface either as a component of a second thinner coating, or broadcast (by hand or spreader) onto an un-set thick epoxy base layers and a second, aesthetically pleasing thin top coat of epoxy comprising finer antimicrobial granules is optionally applied.

Antimicrobial granules made with fine, colored sand are well suited for grouts. Grout so made is highly mildew and mold resistant. The antimicrobial grout is made into a kind of slurry and then squeegeed into the joints with excess being wiped away. For grout, other compounds can be used as appropriate such as cements, latexs, silicones, acrylics and the like, each gaining the antimicrobial attributes of the antimicrobial granules added to it.

Antimicrobial granules have utility as components of mortar as well, for use in brick and tile work, as well. Typically, the epoxy part A resin and part B hardener are mixed with a fine, 100 mesh, anti-microbial silica sand. The amount of antimicrobial silica sand that is added and the specific size of the silica granules allow the epoxy mixed with it to have more of a plaster-type consistency. The actual brick is buttered with the mix, actually placing the mix on the side of the brick. The brick is then set into place. The next brick is also again buttered and they are squeezed together such that the grout can then oozes between and out of the wall formed thereby; the process, therefore, is similar to that used in building a brick wall.

Another method of using antimicrobial filler granules is in the creation of a waterproofing liner mix, useful for example as a waterproof lining for swimming pools or for use in waterproofing basements. This liner mix can be made again with epoxy or other materials into a mix very similar to a drywall compound. This mix can then be used to repair existing walls that are cracked or are otherwise damaged. For such applications, acrylic-based and or latex-based sealant formulations are often used to facilitate cleanup after application. In such cases it can be advantageous to have the granules themselves prepared using a compatible coating system, and one of skill in the art can readily identify such compatible granule coating systems.

As is apparent to those of skill in the art, the applications shown and described are merely examples of some of the uses of the invention of the present application, other uses and examples can be made without departing from the novel scope of the present invention.

Each of the patents and articles cited herein is incorporated by reference. The use of the article “a” or “an” is intended to include one or more.

The descriptions and examples herein are intended as illustrative and are not to be taken as limiting. Still other variations within the spirit and scope of this invention are possible and will readily present themselves to those skilled in the art.

### ILLUSTRATIVE EXAMPLES

### Example 1

### Antimicrobial Granules Coated with a Silver Zeolite Antimicrobialmetal Agent

To coat silica granules and sand granules, a two-part epoxy adhesive was used. Part A was made by mixing 27.8% by weight bisphenol A resin, Epon™ 1001-X-75 (Resolution Performance Products, Houston, Tex.), 36.1% by weight methyl isobutyl ketone, and 36.1% by weight toluene. Part B was made by mixing 50% by weight hardening agent Epikure™ 3115-X-70 (Resolution Performance Products), 25% by weight methyl isobutyl ketone and 25% by weight toluene.

Into a tumbling mixer was placed 50 lbs 50-mesh silica sand with 3% by weight (1.5 lbs) AgIon™ antimicrobial metal agent and, and these were tumbled together for mixing for a minute or two.

To the granule/antimicrobial metal agent mix was added 478 grams of epoxy pre-mix containing about 6 parts by weight Part A and about one part Part B by weight, and the resulting epoxy/granules/antimicrobial mixture was tumbled until the grains were coated and the epoxy had cured or set, about 20-30 minutes, depending on the temperature.

### Example 2

### Antimicrobial Granules Coated with a Silver-Containing Glass Antimicrobial Metal Agent

50 pounds of granite chips, measuring between 1 and 5 mm, are placed in a tumbler and coated as in Example 1, except the antimicrobial metal agent is 1% by weight (0.5 lbs) IONPURE® from Ishizuka Glass Co., Japan. Such granules are appropriate for more ornamental usage than those in Example 1 due to the transparency of the antimicrobial metal agent and the aesthetic appeal of granite.

### Example 3

### Antimicrobial Granules Coated with Elemental Copper, Gold and Silver

50 pounds of glass flakes, RCF-600 from Nippon Glass Sheet Co., Ltd., Japan (available in the U.S. from FRP Services & Co. (America) Inc., White Plains N.Y.) are subjected to electrolytic coating with gold, silver and copper. The method used is the same as for large glass sheets, as is seen on modern office buildings, to give a yellow-green gold color, known in the art as “Gold ON”. While this coating can be applied to flakes, it is most conveniently applied to large sheets of very thin glass that are the intermediate product during manufacture of the glass flakes. Flakes so coated have a coating antimicrobial metal of up to 12 micrometers thick containing 58.5% gold and about 30-35% of a one to one ratio of silver and copper. This process is well understood in the art and is readily adapted to other ratios of antimicrobial metals and deposition of metal oxides such as silver oxide. Such flakes are especially useful for combining chemical and water vapor resistance, electrical conductivity and antimicrobial properties when added to epoxy coatings from approximately 0.5% to about 40% by weight.

### Example 4

### Antimicrobial Granules Coated with Titanium Oxide, Zinc Oxide and Tin Oxide

50 pounds of Provosil-04 (Redco II, North Hollywood, Calif.) perlite is added to a large mixer. To this is added 4 pounds each of titanium oxide, zinc oxide and tin oxide, and the mixer is activated to premix the constituents for 5 minutes. While mixing, 5 gallons of water containing 0.1% surfactant is added to pre-wet the mixture prior to adding the adhesive. 2 gallons of premixed acrylic latex adhesive, comprising 43.17 volumes of NEOCAR Arcylic 850 (Dow Chemical Company, Midland Mich.) 54.58 volumes of water and 2.25 volumes of UCAR® Filmer IBT (Dow Chemical Company, Midland Mich.) is added to the mixer, and mixing continues for 15 minutes, or until the granules are uniformly coated. When uniformly coated, heat is applied while mixing continues, and when the granules have dried, heat is increased to 300° F. for 5 minutes. Alternatively, the coated granules are baked at 300° F. for 5 minutes to complete the curing of the acrylic adhesive. Such antimicrobial granules are Perlite

### Example 5

### Electricity-Conducting Granules Coated with Antimicrobial Glass

To 50 pounds of 100 mesh silica sand in a mixer is added 1.5 pounds of IONPURE® silver-containing glass from Ishizuka Glass Co., Japan, 1.5 pounds of 200 mesh silver powder and 1.5 pounds of carbon black. After premixing for 2 minutes, 1000 grams of the epoxy adhesive of Example 1 is added, and the combination is mixed for 20 minutes to 2 hours until the epoxy hardens. Such granules are especially useful for imparting both electrical conductivity and antimicrobial activity to coatings.

### Example 6

### Epoxy Grout Containing Fine Antimicrobial Granules

Epoxy mortar components Part A and Part B are premixed according to manufacturer's instructions. In place of an equal volume of other fillers, the mortar is then mixed with 20% by weight of fine grained antimicrobial granules prepared from F-80 or 100 mesh fine silica sand, according to the method of Example 1. Such grout retains the beneficial durability, coloring, and other properties of epoxy mortar, but gains the powerful antimicrobial activity of the antimicrobial granules.

I claim:

1. Antimicrobial granules comprising granules that are coated with an antimicrobial amount of an antimicrobial metal agent. and said coating is accomplished by mechanical, chemical, electrical, electrochemical, or adhesive means. The last is a process limitation.

2. The antimicrobial granules of claim 1 wherein said granules are comprised of silica, glass, sand, perlite, aluminum oxide, carborundum, talc, metal silicates, rubber, metal, ceramic, carbon, Kevlar, an ion exchange polymer, and stone.

3. The antimicrobial granule of claim 2 wherein said antimicrobial metal agent comprises at least one metal selected from the group comprising of silver, copper, tin, zinc, titanium and gold.

4. The antimicrobial granule of claim 3 wherein said metal is in an elemental state.

5. The antimicrobial granule of claim 3 wherein said metal is oxidized.

6. The antimicrobial granule of claim 5 wherein said granule is coated by an adhesive.

7. The antimicrobial granule of claim 5 wherein said antimicrobial metal agent further comprises an ion-exchange medium.

8. The antimicrobial granule of claim 7 wherein said ion-exchange medium is selected from the group consisting of a zeolite, a hydroxyapatite, and a glass.

9. A method of imparting antimicrobial characteristics to granules comprising a) providing granules to which antimicrobial properties are to be imparted and b) coating said granules with an antimicrobial metal agent by physical, chemical, electrical or electrochemical means.

10. The method of claim 9 where said granules to which antimicrobial characteristics are to be imparted are comprised of silica, glass, sand, perlite, aluminum oxide, carborundum, talc, metal silicates, rubber, metal, ceramic, carbon, Kevlar, polymer, and stone.

11. The method of claim 10 where said antimicrobial metal agent comprises at least one metal from the group comprising of silver, copper, tin, zinc, titanium and gold.

12. The method of claim 11 where said metal is oxidized.

13. The method of claim 12 where said antimicrobial metal agent further comprises an ion exchange medium.

14. The method of claim 13 where said ion exchange medium is selected from the group consisting of zeolite, a hydroxyapatite, and a glass.

15. The method of claim 14 where said antimicrobial granule is coated with an antimicrobial metal agent by use of an adhesive.

16. The method of claim 15 where said adhesive is selected from the group comprised of epoxies, polyesters, synthetic and natural latexes, acrylics, olefins, vinyls, methacrylates, and urethanes.

17. The method of claim 16 where said adhesive is a two component epoxy and said metal is silver.

18. The method of claim 17 where said granules to which antimicrobial characteristics are to be imparted are between about 10 microns in diameter and about 6 millimeters in diameter.

19. The method of claim 18 where said granules to which antimicrobial characteristics are to be imparted are comprised of sand.

20. The method of claim 18 where said two component epoxy is comprised of a Part A and a Part B, said Part A comprising about 15% to about 50% by weight of an epoxy resin, and about 50% to about 85% by weight of a solvent; said Part B comprising about 25% to about 50% by weigh of a hardening agent and about 50% to about 75% by weight of a solvent.

21. The method of claim 11 where said metal is elemental copper, elemental silver or both elemental copper and elemental silver and said granules to which antimicrobial characteristics are to be imparted are comprised of polymer fibers.

22. The method of claim 12 where said oxidized metal is selected from the group that consists of titanium, zinc, and tin, said granules to which antimicrobial characteristics are to be imparted are comprised of perlite, and said adhesive is an acrylic adhesive.

23. The method of claim 21 where said granule is coated with an antimicrobial metal agent by use of an adhesive, said adhesive being a two component epoxy comprised of a Part A and a Part B, said Part A comprising about 15% to about 50% by weight of an epoxy resin, and about 50% to about 85% by weight of a solvent; said Part B comprising about 25% to about 50% by weigh of a hardening agent and about 50% to about 75% by weight of a solvent.

24. The method of claim 24 where said epoxy resin is a bisphenol A resin.

25. A method of imparting antimicrobial characteristics to a surface by applying to said surface a coating comprising an antimicrobial amount of antimicrobial granules according to claim 1 admixed into a coating base chosen from the group consisting of an epoxy resin, an acrylic, a latex, an olefin, a silicone, a vinylester, Portland cement, a methacrylate, and combinations thereof.

26. The method of claim 25 where said antimicrobial granules comprise about 0.1% to about 90% by weight of said coating.
